(19) United States
(12) Patent Application Publication
(10) Pub. No.: US 2024/0239151 A1
(43) Pub. Date: Jul. 18, 2024

(54) SYSTEM AND METHOD FOR SAMPLING AN AIRPATH WITHIN AN AUTOMOTIVE VEHICLE

(71) Applicant: GM GLOBAL TECHNOLOGY OPERATIONS LLC, Detroit, MI (US)

(72) Inventors: Russell A. Patenaude, Macomb Township, MI (US); Evelyn J. Job, Allenton, MI (US); Karen J. Goodnight, Macomb, MI (US); Cindy Allen, Macomb, MI (US)

(73) Assignee: GM GLOBAL TECHNOLOGY OPERATIONS LLC, Detroit, MI (US)

(21) Appl. No.: 18/097,746

(22) Filed: Jan. 17, 2023

(51) Int. Cl. B60H 1/00; B60H 3/00

(52) U.S. Cl. CPC B60H 1/008; B60H 1/00985; B60H 3/0035

(57) ABSTRACT

A system and method for sampling an airpath within an automotive vehicle are provided. An air sample is obtained from the airpath, and a determination is made whether an air quality of the air sample meets or exceeds a predetermined air quality standard. Air quality data relating to the air sample is transmitted to a back office. Optionally, external route information is received from the back office relating to one or more alternate routes for the vehicle, and a notification is presented within the interior cabin of the one or more alternate routes along with a respective air quality indication for each of the one or more alternate routes.

### INTRODUCTION

This disclosure relates to systems and methods for sampling an airpath within an automotive vehicle, and to systems and methods for collecting and utilizing data from such sampling.

Automotive vehicles have interior cabins into which air is inducted from outside the vehicle. This inducted air is often heated or air conditioned for the comfort of the cabin occupants. However, when the vehicle passes through an area where air pollution, smoke or objectionable smells are in the outside air, this may cause an unpleasant experience for the vehicle's occupants.

### SUMMARY

According to one embodiment, a method for sampling an airpath within an automotive vehicle includes obtaining an air sample from the airpath, wherein the airpath flows from one or more input ports to an interior cabin of the vehicle and then to one or more exit ports, wherein the one or more input ports and the one or more exit ports are in fluid communication with an external environment outside the vehicle. The method further includes determining whether an air quality of the air sample meets or exceeds a predetermined air quality standard, and transmitting air quality data relating to the air sample to a back office.

The method may further include receiving external route information from the back office relating to one or more alternate routes for the vehicle, and presenting a notification within the interior cabin of the one or more alternate routes and of a respective air quality indication for each of the one or more alternate routes, if the air quality fails to meet or exceed the air quality standard.

The air quality data may include one or more of a quantitative indication of the air quality, a qualitative indication of the air quality, and a characterization of one or more components found in the air sample. Additionally, the air quality data may be transmitted to the back office via one or more other automotive vehicles. Further, the notification may be one or both of a visual notification on a display device and an auditory notification over a speaker.

The method may further include, if the air quality fails to meet or exceed the air quality standard, then one or more of: increasing a filtration level of air within the one or more input ports or within the interior cabin;
increasing an odor neutralization level of air within the one or more input ports or within the interior cabin; and releasing a scent within the one or more input ports or within the interior cabin.

The method may further include automatically closing any open windows of the vehicle if the air quality fails to meet or exceed the air quality standard.

The method may further include: (i) if the air quality fails to meet or exceed the air quality standard, then detecting whether an internal fire has occurred onboard the vehicle or an external fire has occurred outside the vehicle; and (ii) if an internal or external fire has been detected, then presenting a warning within the interior cabin regarding the detected internal or external fire. Additionally, if an internal fire has been detected, then internal fire information regarding the internal fire may be received from one or more sensors onboard the vehicle; or, if an external fire has been detected, then external fire information regarding the external fire may be received from one or more external sources outside the vehicle. Moreover, if an internal or external fire has been detected, then emergency services may be automatically notified of the internal or external fire, and fire-related data relating to the internal or external fire may be transmitted to the back office.

The receiving of external route information and the presenting of the notification may be performed if the air quality fails to meet or exceed the air quality standard for at least a predetermined or selected timespan. The method may further include requesting a route selection from among the one or more alternate routes, receiving the route selection, and updating a navigation of the vehicle.

The obtaining of the air sample may be performed when the vehicle is located proximate a predetermined or indicated emission source location, and the method may further include transmitting a reporting flag with the air quality data that relates to the air sample obtained at the predetermined or indicated emission source location.

According to another embodiment, a method for sampling an airpath within an automotive vehicle includes: (i) obtaining an air sample from the airpath, wherein the airpath flows from one or more input ports to an interior cabin of the vehicle and then to one or more exit ports, wherein the one or more input ports and the one or more exit ports are in fluid communication with an external environment outside the vehicle; (ii) determining whether an air quality of the air sample meets or exceeds a predetermined air quality standard; (iii) transmitting air quality data relating to the air sample to a back office, wherein the air quality data includes one or more of a quantitative indication of the air quality, a qualitative indication of the air quality, and a characterization of one or more components found in the air sample; and (iv) if the air quality fails to meet or exceed the air quality standard, then one or more of increasing a filtration level of air within the one or more input ports or within the interior cabin, increasing an odor neutralization level of air within the one or more input ports or within the interior cabin, and releasing a scent within the one or more input ports or within the interior cabin.

According to yet another embodiment, a system for sampling an airpath within an automotive vehicle is provided. In this embodiment, the airpath flows from one or more input ports to an interior cabin of the vehicle and then to one or more exit ports, wherein the one or more input ports and the one or more exit ports are in fluid communication with an external environment outside the vehicle. The system includes: (i) a sensing module configured for obtaining an air sample from the airpath; (ii) a control module in communication with the sensing module and configured for determining whether an air quality of the air sample meets or exceeds a predetermined air quality standard; and (iii) a transmission module in communication with the control module and configured for transmitting air quality data relating to the air sample to a back office.

The system may further include: (iv) a reception module in communication with the control module and configured for receiving external route information from the back office relating to one or more alternate routes for the vehicle; and (v) a presentation module in communication with the control module and configured for presenting a notification within the interior cabin of the one or more alternate routes and of a respective air quality indication for each of the one or more alternate routes.

The control module may include a processor and a memory in communication with the processor, with the memory containing an instruction set operable to: cause the sensing module to obtain the air sample from the airpath; cause the processor to determine whether the air quality of the air sample meets or exceeds the predetermined air quality standard; and cause the transmission module to transmit air quality data relating to the air sample to the back office.

The sensing module may be further configured for detecting whether an internal fire has occurred onboard the vehicle and the instruction set may be further operable to cause the transmission module to notify emergency services of the internal fire, and cause the transmission module to transmit fire-related data relating to the internal fire to the back office, if an internal fire onboard the vehicle has been detected.

The system may further include an odor mitigation module in communication with the control module and configured for one or more of increasing a filtration level of air within the one or more input ports or within the interior cabin, increasing an odor neutralization level of air within the one or more input ports or within the interior cabin, and releasing a scent within the one or more input ports or within the interior cabin.

The instruction set may be further operable to cause the sensing module to obtain the air sample when the vehicle is located proximate a predetermined or indicated emission source location, and cause the transmission module to transmit a reporting flag with the air quality data that relates to the air sample obtained at the predetermined or indicated emission source location.

The above features and advantages, and other features and advantages, of the present teachings are readily apparent from the following detailed description of some of the best modes and other embodiments for carrying out the present teachings, as defined in the appended claims, when taken in connection with the accompanying drawings.

### BRIEF DESCRIPTION OF THE DRAWINGS

FIG. 1 is a schematic plan view of an automotive vehicle for use with the system and method of the present disclosure.

FIG. 2 is a network diagram of the subject vehicle of FIG. 1 along with other vehicles and elements in an external environment outside the subject vehicle.

FIG. 3 is a block diagram of a system for sampling an airpath within the subject vehicle.

FIG. 4 is a block diagram of a controller/control module for use in the system of FIG. 3.

FIG. 5 is a block diagram of various types of fires which the system and method may detect.

FIG. 6 is a block diagram of the airpath and various flows and relationships of data, information and signals stemming from a sampling of the airpath.

FIG. 7 is a flowchart of actions according to a method for sampling an airpath within the subject vehicle.

### DETAILED DESCRIPTION

Referring now to the drawings, wherein like numerals indicate like parts in the several views, a system 42 and method 100 for sampling an airpath 28 within a subject automotive vehicle 10 are shown and described herein. The system 42 and method 100 are also useful for collecting and utilizing various data from sampling the airpath 28 within the subject vehicle 10.

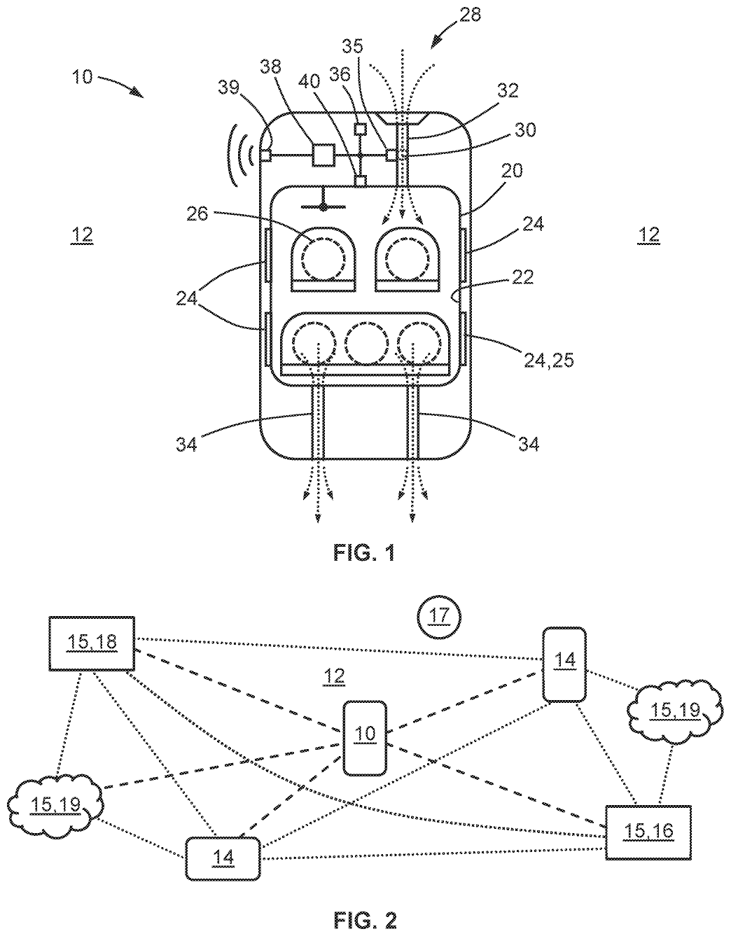

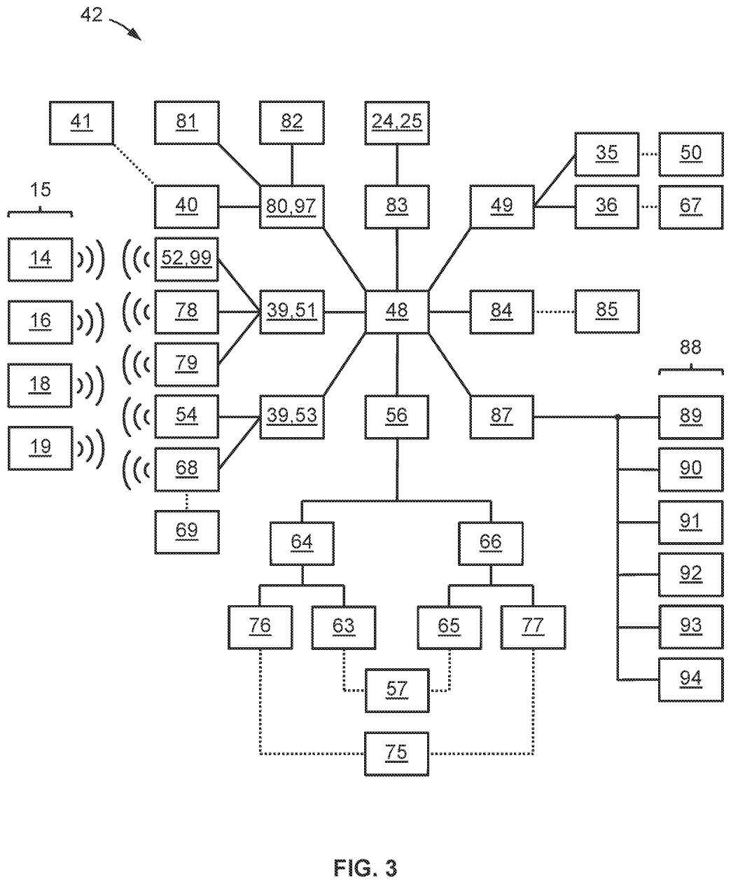

FIG. 1 shows a schematic plan view of a subject vehicle 10 which may be used with the system 42 and method 100, FIG. 2 shows a network diagram of the vehicle 10 along with other vehicles 14 and elements in an external environment 12 outside the subject vehicle 10, and FIG. 3 shows a block diagram of the system 42.

FIG. 1 shows the subject vehicle 10 in isolation, whereas FIG. 2 shows the subject vehicle 10 along with other elements that are within the subject vehicle's external environment 12. These other elements include other automotive vehicles 14 as well as various external sources 15 of information and data. These external sources 15 may include a back office 16 (e.g., OnStar®), various emergency services 18 (e.g., police, firefighters, rescue units, ambulances, animal control units, etc.), and various other external data and information sources 19 (e.g., the National Oceanic and Atmospheric Administration (NOAA), the National Weather Service (NWS), the Federal Emergency Management Agency (FEMA), weather.com, etc.) including the cloud/internet. The back office 16 may comprise a single site or it may comprise a distributed network of sites. In FIG. 2, the dashed lines represent lines of communication between the subject vehicle 10 and each of the other vehicles 14 and external sources 15, while the dotted lines represent lines of communication among the other vehicles 14 and external sources 15. These lines of communication may include transmission and/or reception, and may utilize various electromagnetic frequencies (e.g., radio frequencies) and communication protocols.

The external environment or atmosphere 12 outside the vehicle 10 may include one or more emission source locations 17, which may be sites or areas that have been indicated or reported (e.g., by the back office 16 or other external sources 15, or by an occupant 26 in the vehicle 10 or in another vehicle 14), or which are predetermined or previously known, as having a heightened or unacceptable level of air pollution, smog or other undesirable odors or emissions. For example, such emission source locations 17 may include garbage dumps, refineries, certain chemical or industrial factories, burning buildings, active wildfire zones, and areas of dense, slow-moving traffic where tailpipe emissions from internal combustion engines may be accumulated.

In FIG. 1, the subject vehicle 10 includes an interior cabin 20 which is defined by various interior walls and surfaces 22, such as the front and rear windshields, the side windows 24, the headliner, the floorboard, the front dashboard, the doors, the A-, B- C- and D-pillars, etc. As illustrated in the drawing, the right-rear window is shown as an open window 25, but any, all or none of the windows 24 may be an open window 25 at any given time. The dashed circles represent seating positions for occupants 26 within the interior cabin 20.

An airpath 28 (i.e., a flow path for air, denoted by the dotted arrow lines) is shown entering at the front of the vehicle 10 (e.g., at an air scoop or air intake) and being directed by one or more input ports 30 into the interior cabin 20, and directed by one or more exit ports 32 out of the interior cabin 20. Although the drawing illustrates the use of one input port 30 and two exit ports 32, any number of input and exit ports 30, 32 may be used. Additionally, air may flow into and out of the interior cabin 20 by other means as well; in such a case, each path of air inflow may constitute an input port 30 and each path of air outflow may constitute an exit port 32.

An air sample 34 is shown as a volume or region of air within an input port 30 which may be sampled by an airpath sensor 35. For example, the airpath sensor 35 may be a chemical sensor capable of sensing the chemical characteristics of the air sample 34, a particulate sensor capable of sensing the particulate characteristics of the air sample 34, or the like. It may be noted that while the air sample 34 is shown as being drawn from an input port 30, the sample 34 may also be drawn from an output port 32 or from within the interior cabin 20. A single internal fire sensor 36 is also shown within the front of the vehicle 10, but two or more internal fire sensors 36 may also be used, with each sensor 36 being positioned at a suitable location within the vehicle 10. For example, the internal fire sensor 36 may be capable of sensing the thermal, chemical and/or other characteristics of its immediate environment, such that it is capable of detecting the presence of an internal fire 37 within the vehicle 10 (such as inside the engine bay, around a battery pack, etc.).

Each airpath sensor 35 and internal fire sensor 36 may be operatively connected to a controller 38 which is configured for receiving signals from the sensors 35, 36. The controller 38 may also be operatively connected with a transceiver 39, such that the transceiver 39 may receive signals/information from the back office 16, other vehicles 14 and/or various external sources 15 and share such signals/information with the controller 38, and such that the controller 38 may share signals/information with the transceiver 39 so as to cause the transceiver 39 to transmit such signals/information to the back office 16, other vehicles 14 and/or various external sources 15.

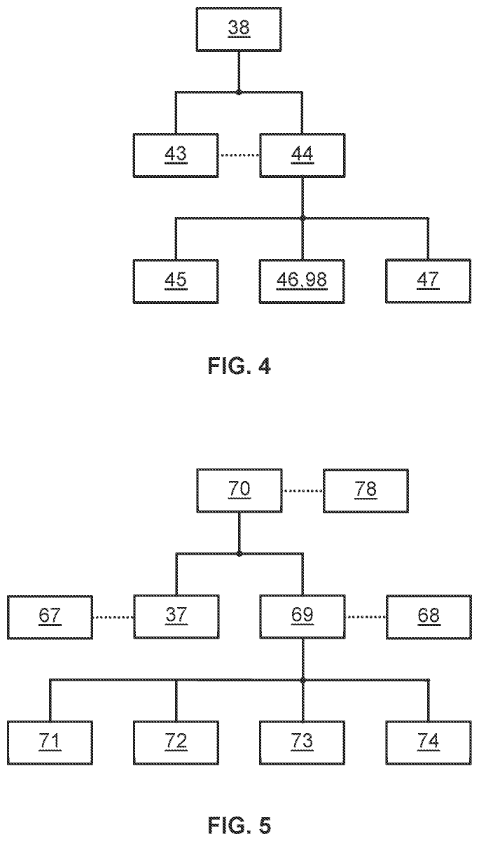

FIG. 3 shows a block diagram of the system 42 for sampling the airpath 28 within a vehicle 10. The system 42 includes a control module 48, and as shown in the block diagram of FIG. 4, the control module 48 may include or comprise the abovementioned controller 38. The controller 38 may include a processor 43 operatively connected with a memory 44, wherein the memory 44 may include or be configured to store an instruction set 45, one or more parameters 46 and an air quality standard 47. For example, the instruction set 45 may comprise software or code that is executable by the processor 43, the parameters 46 may be values associated with respective variables in the code/instruction set 45 (such as a timespan 98 that has been factory pre-set or has been set by an occupant 26), and the air quality standard 47 may comprise predetermined values that are particularly related to one or more acceptance standards for air quality (e.g., for the acceptable levels of various chemicals and/or particulates that may be found in atmospheric air). The air quality standard 47 may be pre-loaded into a portion of the memory 44 (e.g., in a look-up table) or it may be received into the controller 38 and memory 44 from an external source 15 (e.g., from the back office 16) via the transceiver 39. Similarly, the one or more parameters 46 may also be pre-loaded into a portion of the memory 44 (e.g., in one or more registers) or they may be received into the controller 38 and memory 44 from an external source 15 via the transceiver 39. Optionally, one or more of the parameters 46 may be entered into the controller 38 and memory 44 by an occupant 26 or service technician via input module 87 or input device 88, as described in further detail below.

The control module/controller 48, 38 is configured for determining whether an air quality 50 of the air sample 34 meets or exceeds the predetermined air quality standard 47. This determination may be performed by the control module/controller 48, 38 by comparing various qualitative and/or quantitative characteristics of the air quality 50 to corresponding values in the air quality standard 47. For example, the control module/controller 48, 38 may compare the ozone level, sulfurous compound level, particle count and average particle size for a given volume of the air sample 34 against the respective permissible levels for these characteristics in the air quality standard 47. After this comparison or determination is made, a set of air quality data 52 may be produced which represents the result of this comparison or determination. Such air quality data 52 may include pass/fail indications (e.g., “1” or “P” for pass, “0” or “F” for fail), qualitative indications (e.g., a letter or number representing “poor”, “marginal”, “good”, “excellent”, etc.) and/or quantitative/numerical indications for each comparison (e.g., for the ozone level, the sulfurous compound level, the particle count and the average particle size).

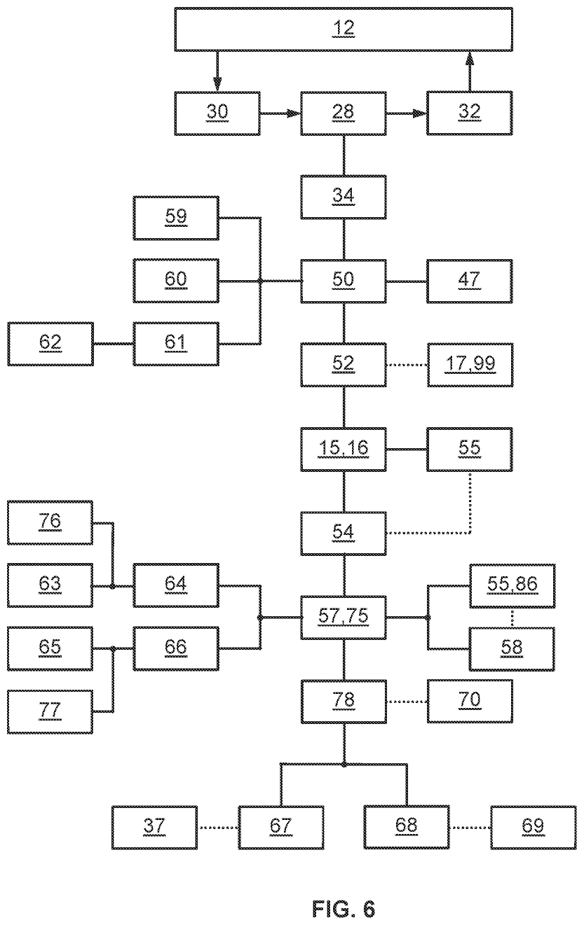

In addition to the control module 48, the system 42 may also include a sensing module 49 in communication with the control module 48 and configured for obtaining an air sample 34 from the airpath 28, a transmission module 51 in communication with the control module 48 and configured for transmitting the air quality data 52 to the back office 16 or other external sources 15, a reception module 53 in communication with the control module 48 and configured for receiving external route information 54 from the back office 16 or other external sources 15 relating to one or more alternate routes 55 for the vehicle 10, and a presentation module 56 in communication with the control module 48 and configured for presenting a notification 57 within the interior cabin 20 of the vehicle 10 of the one or more alternate routes 55 and of a respective air quality indication 58 for each alternate route 55. These and other modules which may be included in the system 42 will now be described in more detail, in conjunction with FIG. 3 (showing the system 42) and FIG. 6 (showing a block diagram of the airpath 28 and various flows and relationships of data, information and signals stemming from the sampling of the airpath 28).

The sensing module 49 may include or comprise the airpath sensor 35, as well as the one or more internal fire sensors 36. For example, the airpath sensor 35 may provide the sensing module 49 with data or characteristics relating to the air quality 50 of the air sample 34, so that the control module 48 may compare these data or characteristics against the air quality standard 47. Additionally, the one or more internal fire sensors 36 may sense or detect whether an internal fire 37 has occurred within the vehicle 10 (e.g., in the immediate vicinity around a sensor 36), and may alert the control module 48 via an appropriate signal (containing or relating to internal fire information 67) if an internal fire 37 has been detected.

The transmission module 51 may include or comprise the transceiver 39 or a transmitting portion thereof. Similarly, the reception module 53 may include or comprise the transceiver 39 or a receiving portion thereof. In some configurations, the transceiver 39 may include or comprise both the transmission module 51 and the reception module 53. The transmission module 51 may transmit air quality data 52, fire-related data 78, emergency services requests/notifications 79, and the like to one or more external services 15, such as the back office 16, emergency services 18, other vehicles 14 or other external sources 19. The reception module 53 may receive external route information 54, external fire information 68 (i.e., information about external fires 69 outside the vehicle 10 that have been reported to the authorities), and the like from one or more external services 15, such as the back office 16, emergency services 18, other vehicles 14 or other external sources 19.

The external route information 54 may relate to possible alternate routes 55 which the vehicle 10 may take in order to avoid an area of poor or objectionable air quality 50 that the vehicle 10 may currently be driving through. For example, these alternate routes 55 may have an air quality 50 that is better or more desirable than the air quality 50 of the vehicle's current location or of an upcoming location which the vehicle's navigation path goes through or near, such as a predetermined or indicated emission source location 17.

When the back office 16 receives air quality data 52 from the control module 48 (via the transceiver 39) which indicates that the air quality 50 of the air sample 34 has failed to meet or exceed the air quality standard 47, the back office 16 may then provide or broadcast external route information 54, which may be received by the reception module 53 and shared with the control module 48. As described in further detail below, the control module 48 may then utilize this information, along with a route selection 86 made by an occupant 26, to cause a navigation module 84 to alter the navigation 85 of the vehicle 10 to follow the selected alternate route 55. Optionally, the back office 16 may provide or broadcast external route information 54 for possible alternate route 55 without receiving air quality data 52 that indicates a poor air condition, such as to avoid a predetermined or indicated emission source location 17.

The abovementioned external fire information 68, as further illustrated in FIG. 5, may relate to external fires 69 that are grass fires 71, other vehicle fires 72, building structure fires 73 or other types of fires 74. Thus, the external fire information 68 may include an identification of which type of external fire 69 has been reported. In one example, the control module 48 may receive external fire information 68 from the back office 16 via the reception module 53, and may transmit some or all of the external fire information 68 as fire-related data 78 to other vehicles 14 via the transmission module 51. In another example, the control module 48 may transmit internal or external fire information 67, 68 as fire-related data 78 to other nearby vehicles 14 via the transmission module 51 if the back office 16 is unavailable (e.g., off-line or out of signal range), the other vehicles 14 may relay this information to local emergency services 18, the local emergency services 18 may respond to the fire and relay the information (and the fact that it is responding to the detected fire 70) to the back office 16, and the back office 16 may further relay or broadcast this information as external fire information 68, which may be received by the control module 48 via the reception module 53. Many other related scenarios are possible as well.

Note that FIG. 3 shows signals being transmitted and received by the transmission and reception modules 51, 53, respectively, as well as by the various external sources 15. Optionally, the transmission and reception modules 51, 53 may be predesignated to communicate preferentially or exclusively with selective portions of the external sources 15, or such designations may be determined, selected or assigned on-the-fly in real time. For example, the transmission module 51 may be designated or caused to communicate certain types of information according to a particular hierarchy, order or priority plan (e.g., to back office 16 first, then to other vehicles 14, etc.), while other types of information may be transmitted according to a different hierarchy or order. Similarly, the reception module 53 may be designated or caused to receive certain types of information according to a particular hierarchy, order or priority plan (e.g., external route information 54 may only be received from the back office 16, while external fire information 68 may be received first from the back office 16, then emergency services 18, etc.). The priority plan for the reception module 53 may also include a designation as to which of the external sources 15 take precedence over others and in what order; for example, the priority plan may assign top priority or preference to any information from the back office 16, then to information received from emergency services 18, then to other external sources 19 besides other vehicles 14, and finally to other vehicles 14. Optionally, the control plan may apply to all types of received information, or it may vary depending upon the specific type of received information.

The presentation module 56 may include one or more display devices 64 and/or one or more speakers 66 for presenting notifications 57 to the occupants 26 within the interior cabin 20. The notifications 57 may be presented as a visual notification 63 on a display device 64 (e.g., as an icon, symbol or text, which optionally may be flashing or color-coded) and/or as an auditory notification 65 through a speaker 66 (e.g., as orated text or as an alarm/alert). The notification 57 may convey information relating to the one or more alternate routes 55 for the vehicle 10, as well as a respective air quality indication 58 for each of the one or more alternate routes 55. For example, a display device 64 may show three alternate routes 55 which may be selected or taken, with an indication 58 of the respective air quality for each of the three routes 55. For instance, the air quality indications 58 may be presented on a numerical scale of 1 to 100, or they may be color-coded (e.g., red, yellow, green) or given a qualitative designation (e.g., “Poor”, “Marginal”, “Acceptable”), or any combination of the foregoing. Optionally, the notification 57 may also include an air quality indication 58 for the current route that the vehicle 10 is on, in order for an occupant 26 to compare the air quality indication 58 of the alternate routes 55 with the current route.

The system 42 may also include an input module 87 in communication with the control module 48 and configured for receiving input from an occupant 26 within the interior cabin 20 of the vehicle 10. The input module 87 may include one or more input devices 88, such as a keyboard 89, touchscreen 90, button 91, switches/toggles 92, dials 93, a microphone 94, or the like. The input module/input devices 87, 88 may be used by an occupant 26 to provide inputs to the control module 48, such as in response to a question, alert or prompt triggered by the control module 48 via the presentation module 56 (e.g., via a display device/screen 64 and/or via a speaker 66). For example, the control module 48 may cause the presentation module 56 to present a request for a route selection 86 along with the alternate route information 54 that is presented, and in response the input module 87 may be used by an occupant 26 to select or input their route selection 86, which the input module 87 will then share with the control module 48; the control module 48 may then utilize this route selection 86 to cause a navigation module 84 to follow the selected alternate route 55, as described in further detail below.

The system 42 may further include an odor mitigation module 80 in communication with the control module 48 and configured for one or more of increasing a filtration level 81 of air that is within the one or more input ports 30 or within the interior cabin 20, increasing an odor neutralization level 82 of air that is within the one or more input ports 30 or within the interior cabin 20, and releasing a scent 41 within the one or more input ports 30 or within the interior cabin 20. For example, the odor mitigation module 80 may include or comprise a mechanical filter, a chemical filter and a scent dispenser 40, wherein the control module 48 is configured to cause the air within the one or more input ports 30 and/or within the interior cabin 20 to be treated by one or more of the mechanical filter, the chemical filter and the scent dispenser 40 (such as by directing the air through or past the mechanical/chemical filters and/or by releasing a scent 41 into the air).

The filtration level 81 may be provided by a mechanical filter having a porous medium or membrane through which air may be directed. The filtration level 81 of the air within the one or more input ports 30 and/or within the interior cabin 20 may be increased by passing the air through the mechanical filter if it was not previously passing therethrough, or by passing the air through a greater volume of the filter or through one or more additional mechanical filters. The odor neutralization level 82 may be provided by a chemical or reactive filter having surfaces or pores treated with one or more of a deodorizer, an odor neutralizer and an odor blocker. (For example, the chemical/reactive filter may include embedded particles of activated carbon, silver or the like.) The odor neutralization level 82 being increased by passing the air through or along the chemical/reactive filter if it was not previously passing therethrough or therealong, or by passing the air through a greater volume or along a greater surface area of the chemical/reactive filter or through/along one or more additional chemical/reactive filters. The scent 41 may be a pleasant-smelling fragrance in a mist or atomized form.

The system 42 may additionally include a navigation module 84 in communication with the control module 48 and configured for operating with a navigation plan or navigation data (collectively referred to herein as a “navigation” 85) for controlling the speed and direction (i.e., the acceleration, braking and steering) of the vehicle 10. If the control module 48 has determined that the air quality 50 of the air sample 34 has not met or exceeded the air quality standard 47, and if the control module 48 has received external route information 54 (relating to one or more alternate routes 55) via the reception module 53, the control module 48 may present a notification 57 to an occupant 26 via the presentation module 56 regarding the alternate routes 55 and their respective air quality indications 58. The control module 48 may then receive a route selection 86 from the occupant 26 via the input module 87, and may then cause the navigation module 84 to adjust its navigation 85 based on the occupant's selection.

The system 42 may further include a window module 83 in communication with the control module 48 and configured for automatically closing any open windows 25 of the vehicle 10 if the air quality 50 fails to meet or exceed the air quality standard 47. (As used here, closing any open windows 25 “automatically” means that the open windows 25 are closed by an electromechanical mechanism without direct action by an occupant 26). Any open windows 25 may be automatically closed by the window module 83 in order to minimize the exposure of the interior cabin 20 to air from the external environment 12 through the windows 24, 25.

The instruction set 45 of the control module/controller 48, 38 may optionally include instructions to cause the sensing module 49 to obtain the air sample 34 when the vehicle 10 is located proximate a predetermined or indicated emission source location 17, and to cause the transmission module 51 to transmit a reporting flag 99 with the air quality data 52 that relates to the air sample 34 obtained at the emission source location 17. For example, the reporting flag 99 may be a unique sequence of bits, symbols or words that function to mark the presence of air quality data 52 that relates to the air sample 34 obtained in the vicinity of the emission source location 17. The inclusion of such a reporting flag 99 along with a stream of air quality data 52 may serve to identify the particular portion of the stream which relates to the air sample 34 obtained at the emission source location 17; for example, the reporting flag 99 may be inserted into the stream of air quality data 52 immediately after an air sample 34 is taken at the emission source location 17. Optionally, whenever an occupant 26 utilizes the input module 87 to indicate that an emission source location 17 is being traveled through (e.g., the vehicle 10 passes by an external fire 69 and smoke or other noxious fumes are smelled by the occupant 26), the control module 48 may designate the vehicle's current location as an emission source location 17 and may optionally transmit this information/designation to the back office 16 and/or to other external sources 15 (such as to emergency services 18).

When an internal fire sensor 36 detects an internal fire 37 within the vehicle 10 (e.g., within the engine bay, near a battery pack, near an electric traction motor, etc.) and sends internal fire information 67 to the control module 48, and/or when the control module 48 receives external fire information 68 from an external source 15 (e.g., the back office 16) via the reception module 53 relating to an external fire 69, the control module 48 may cause the presentation module 56 to provide a warning 75 to the occupants 26 of the detected internal or external fire 37, 69. This warning 75 may be provided or presented as a visual warning 76 on a display device 64 and/or as an auditory warning 77 through a speaker 66.

As used herein, a “module” may include hardware and/or software, including executable instructions, for receiving one or more inputs, processing the one or more inputs, and providing one or more corresponding outputs. (For example, these one or more inputs may include signals or conditions that indicate or are the result of an action which the module is configured for, such as transmitting, receiving, presenting, etc., or an event or condition which the module is configured for detecting or sensing.) Also note that at some points throughout the present disclosure, reference may be made to a singular input, output, element, etc., while at other points reference may be made to plural/multiple inputs, outputs, elements, etc. Thus, weight should not be given to whether the input(s), output(s), element(s), etc. are used in the singular or plural form at any particular point in the present disclosure, as the singular and plural uses of such words should be viewed as being interchangeable, unless the specific context dictates otherwise.

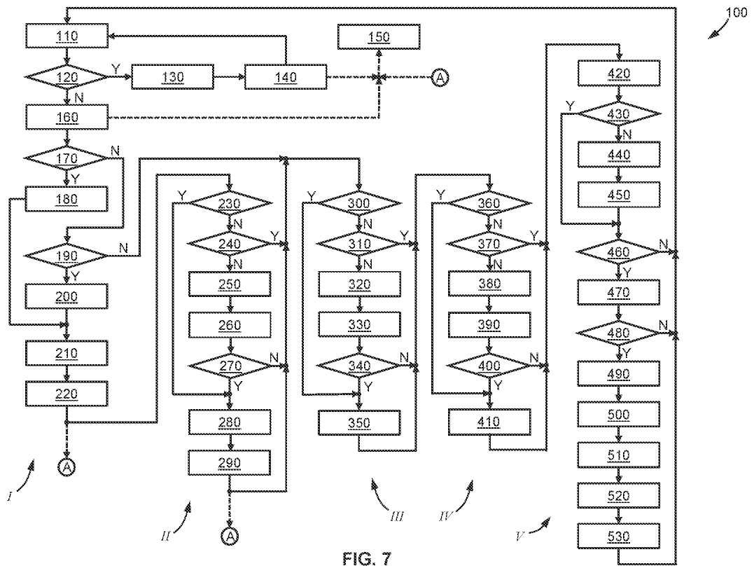

The system 42 of the present disclosure may be utilized to carry out the abovementioned method 100 for sampling an airpath 28 within a subject vehicle 10. FIG. 7 shows a flowchart of actions according to the method 100.

At block 110, an air sample 34 is obtained from the airpath 28, and at block 120, it is determined whether an air quality 50 of the air sample 34 meets or exceeds a predetermined air quality standard 47. If the air quality 50 of the air sample 34 does meet or exceed the air quality standard 47, then, at block 130, a timer or loop counter is reset or unlatched, and at block 140 the air quality 50 information is sent to the back office 16, and the process flow is directed back to block 110 where the air is sampled again. Optionally, the air quality 50 information may also be used at block 150 (e.g., by the back office 16) to re-route other vehicles 14 and traffic; for example, if other vehicles 14 are going through an area of poor or objectionable air quality, then they may be redirected through the area where the subject vehicle 10 has indicated that the air quality 50 thereat has met or exceeded the air quality standard 47.

If the air quality 50 of the air sample 34 does not meet or exceed the air quality standard 47, then, at block 160, the air quality data 52 relating to the air sample 34 is sent to the back office 16 (similar to block 140), and at block 170, a first branch/of the process flow may be entered. At block 170, a detection is made (e.g., by the one or more internal fire sensors 36) whether an internal fire 37 has occurred onboard the vehicle 10. If an internal fire 37 has been detected, then, at block 180, internal fire information 67 is received (e.g., by the controller/control module 38, 48) regarding the internal fire 37 from the one or more sensors 36, and the process flow is then directed to block 210; however, if no internal fire 37 has been detected, then the process flow is directed to block 190. At block 190, a detection is made (e.g., by the controller/control module 38, 48 via the reception module 53) whether an external fire 69 has occurred outside the vehicle 10. If an external fire 69 has been detected, then, at block 200, external fire information 68 is received (e.g., by the controller/control module 38, 48) regarding the external fire 69 from the back office 16 and/or from other external sources 15, and the process flow is then directed to block 210; however, if no external fire 69 has been detected, then the process flow is directed to block 300.

Note that while FIG. 7 shows the process flow skipping block 190 (i.e., checking for an external fire 69) if an internal fire 37 has been detected at block 170, in some configurations the method 100 may avoid this skipping and may include checking for an external fire 69 even if an internal fire 37 has been detected. Also note that as used herein, the words “detecting”, “determining”, “checking”, “interrogating”, “querying”, and their associated prepositions and subordinators (“for”, “whether”, “if”, etc.), along with their various word forms and active/passive voice forms (e.g., detecting, detection, detect, is detected), may be used interchangeably, unless otherwise indicated by context. Further, note that as used herein, the words “transmitting” and “sending”, along with their various word forms (e.g., transmitting, transmission, transmit, is transmitted), may be used interchangeably, unless otherwise indicated by context.

At block 210, a warning 75 is presented (e.g., via the presentation module 56) regarding the detected internal or external fire 37, 69, and at block 220, internal fire information 67 and/or external fire information 68 is transmitted (e.g., by the control module 48 and transmission module 51) to the back office 16. The process flow is then directed to block 230, and optionally may also be directed to jump point or connector “A”. Note that jump point “A” may also feed the internal/external fire information 67, 68 into block 150, where this information 67, 68 may be utilized to re-route other traffic.

At block 230, a second branch II of the process flow may be entered. At block 230, a determination is made whether an automatic emergency services 18 notification feature has been predetermined, preset or previously selected (e.g., by an occupant 26 or service technician) to be “ON”; if so, then the process flow is directed to block 280, but if not, then the process flow is directed to block 240. At block 240, a determination is made whether the automatic emergency services 18 notification feature has been predetermined, preset or previously selected to be “OFF”; if so, then the process flow is directed to block 300, but if not, then the process flow is directed to block 250. At block 250, a query, prompt or interrogation is presented to the occupants 26 within the interior cabin 20 (e.g., via the presentation module 56) whether to set the automatic emergency services 18 notification feature to be “ON”, and at block 260, a response to the query, prompt or interrogation is received (e.g., via an input module/input device 87, 88). At block 270, a determination is made whether the response is for the automatic emergency services 18 notification feature to be “ON”; if so, then the process flow is directed to block 280, but if not, then the process flow is directed to block 300. At block 280, emergency services 18 are automatically notified (e.g., via the transmission module 51 and/or the back office 16) of the internal and/or external fire 37, 69, so that appropriate emergency personnel and equipment may be sent, and at block 290, the fact that emergency services 18 has been contacted is sent to the back office 16. The process flow is then directed to block 300, and optionally may also be directed to jump point or connector “A” and from there to block 150, where the fact that emergency services 18 has been contacted (and presumably will be dispatched) may be utilized to re-route other traffic.

At block 300, a third branch III of the process flow may be entered. At block 300, a determination is made whether an open window 25 auto-closing feature has been predetermined, preset or previously selected to be “ON”; if so, then the process flow is directed to block 350, but if not, then the process flow is directed to block 310. At block 310, a determination is made whether the open window 25 auto-closing feature has been predetermined, preset or previously selected to be “OFF”; if so, then the process flow is directed to block 360, but if not, then the process flow is directed to block 320. At block 320, a query, prompt or interrogation is presented to the occupants 26 within the interior cabin 20 (e.g., via the presentation module 56) whether to set the open window 25 auto-closing feature to be “ON”, and at block 330, a response to the query, prompt or interrogation is received (e.g., via an input module/input device 87, 88). At block 340, a determination is made whether the response is for the open window 25 auto-closing feature to be “ON”; if so, then the process flow is directed to block 350, but if not, then the process flow is directed to block 360. At block 350, any open windows 25 are automatically closed (e.g., via the window module 83) of the internal and/or external fire 37, 69, so that no further poor quality air may enter the interior cabin 20 through the previously open windows 25, and the process flow is then directed to block 360.

At block 360, a fourth branch IV of the process flow may be entered. At block 360, a determination is made whether odor mitigation measures 97 have been predetermined, preset, previously selected to be “ON”; if so, then the process flow is directed to block 410, but if not, then the process flow is directed to block 370. At block 370, a determination is made whether the odor mitigation measures 97 have been predetermined, preset or previously selected to be “OFF”; if so, then the process flow is directed to block 420, but if not, then the process flow is directed to block 380. At block 380, a query, prompt or interrogation is presented to the occupants 26 within the interior cabin 20 (e.g., via the presentation module 56) whether to set the odor mitigation measures 97 to be “ON”, and at block 390, a response to the query, prompt or interrogation is received (e.g., via an input module/input device 87, 88). At block 400, a determination is made whether the response is for the odor mitigation measures 97 to be “ON”; if so, then the process flow is directed to block 410, but if not, then the process flow is directed to block 420. At block 410, one or more of the odor mitigation measures 97 are performed (e.g., via the odor mitigation module 80). Note that the above steps of the fourth branch IV may apply to all three of the odor mitigation measures 97 together—i.e., to increasing the filtration level 81, increasing the odor neutralization level 82, and releasing a pleasant-smelling scent 41—or the steps of the fourth branch IV may be duplicated and applied to one or two of the three odor mitigation measures 97 at a time.

At block 420, a fifth branch V of the process flow may be entered. At block 420, the aforementioned timer or loop counter may be latched or set (if it is not already latched or set), so that a timespan 98 or a number of loops through the process flow may be counted. At block 430, a determination is made whether the timespan 98 has been predetermined, preset, previously selected; if so, then the process flow is directed to block 460, but if not, then the process flow is directed to block 440. At block 440, a query, prompt or interrogation is presented to the occupants 26 within the interior cabin 20 (e.g., via the presentation module 56) asking to enter or select a number of minutes for the timespan 98, and at block 450, a response to the query, prompt or interrogation is received (e.g., via an input module/input device 87, 88). (The control module 48 may optionally translate the entered number of minutes into a number of process loops, where each process loop comprises one pass through any of the first through fifth branches I-V. For example, this may be determined as a function of a clock speed of the processor 43, a capacity or bandwidth of the processor 43, and a current or estimated load on the processor 43.)

At block 460, a determination is made whether the latched timer has met or exceeded the timespan 98; if so, then the process flow is directed to block 470, but if not, then the process flow is directed to block 110. At block 470, external route information 54 may be received (e.g., via the reception module 53) from the back office 16 relating to one or more alternate routes 55 for the subject vehicle 10. At block 480, a determination is made whether at least one alternate route 55 has been received; if so, then the process flow is directed to block 490, but if not, then the process flow is directed back to block 110.

At block 490, a notification 57 is presented (e.g., via the presentation module 56) of the alternate routes 55 and their respective air quality indications 58, at block 500, a route selection 86 is requested (e.g., also via the presentation module 56) from among the one or more alternate routes 55, and at block 510, a route selection 86 is received (e.g., via the input module 87). For example, an occupant 26 may see alternate routes 55 and their air quality indications 58 displayed on a display device 64 which is also a touchscreen 90, and the occupant 26 may make a route selection 86 from among the displayed alternate routes 55 via the touchscreen 90, based on comparing the air quality indications 58 of the routes 55.

At block 520, the navigation 85 of the vehicle 10 (e.g., the vehicle's speed and trajectory) may be updated and modified utilizing the route selection 86. For example, the navigation 85 may re-route the vehicle 10 away from a current location where the air quality 50 of the air sample 34 has failed to meet or exceed the air quality standard 47, and/or away from an oncoming emission source location 17, and toward a route having a better air quality as suggested by the air quality indication 58 of the selected alternate route 55. Finally, at block 530, the timer may be unlatched or reset if it was already latched or set, and the process flow is directed back to block 110.

It may be noted that the first through fifth branches I-V are only available for execution if the air quality 50 of the air sample 34 fails to meet or exceed the predetermined air quality standard 47. If the air quality 50 of the air sample 34 does meet or exceed the standard 47, then the method 100 will continue to sample the airpath 28 until such time as the air quality 50 of the air sample 34 fails to meet or exceed the standard 47.

Further, it may be noted that selected actions or portions of the process flow of the method 100 may be subject to a previous selection or authorization, such as by an occupant 26, as a factory preset, as a software/firmware update, by a technician or service person at a previous servicing of the vehicle, or the like. For example, the automatic emergency services 18 notification feature in the second branch II, the open window 25 auto-closing feature in the third branch III, and the performance of one or more of the odor mitigation measures 97 in the fourth branch IV may each be performed if previously selected or authorized.

Various embodiments and configurations of the system 42 and method 100 may be utilized for sampling the airpath 28 within a subject vehicle 10.

For example, according to one embodiment, a method 100 for sampling an airpath 28 within an automotive vehicle 10 includes, at block 110, obtaining an air sample 34 from the airpath 28, wherein the airpath 28 flows from one or more input ports 30 to an interior cabin 20 of the vehicle 10 and then to one or more exit ports 32, wherein the one or more input ports 30 and the one or more exit ports 32 are in fluid communication with an external environment 12 outside the vehicle 10. The method 100 further includes: at block 120, determining whether an air quality 50 of the air sample 34 meets or exceeds a predetermined air quality standard 47; and at block 160, transmitting air quality data 52 relating to the air sample 34 to a back office 16.

The method 100 may further include, at block 470, receiving external route information 54 from the back office 16 relating to one or more alternate routes 55 for the vehicle 10, and, at block 490, presenting a notification 57 within the interior cabin 20 of the one or more alternate routes 55 and of a respective air quality indication 58 for each of the one or more alternate routes 55, if the air quality 50 fails to meet or exceed the air quality standard 47.

The air quality data 52 may include one or more of a quantitative indication 59 of the air quality 50, a qualitative indication 60 of the air quality 50, and a characterization 61 of one or more components 62 found in the air sample 34. Additionally, the air quality data 52 may be transmitted to the back office 16 via one or more other automotive vehicles 14. Further, the notification 57 may be one or both of a visual notification 63 on a display device 64 and an auditory notification 65 over a speaker 66.

The method 100 may further include, at block 410, performing one or more odor mitigation measures 97 if the air quality 50 fails to meet or exceed the air quality standard 47. These odor mitigation measures 97 may include one or more of: (i) increasing a filtration level 81 of air within the one or more input ports 30 or within the interior cabin 20; (ii) increasing an odor neutralization level 82 of air within the one or more input ports 30 or within the interior cabin 20; and (iii) releasing a scent 41 within the one or more input ports 30 or within the interior cabin 20.

The method 100 may further include, at block 350, automatically closing any open windows 25 of the vehicle 10 if the air quality 50 fails to meet or exceed the air quality standard 47.

In this method 100, if the air quality 50 fails to meet or exceed the air quality standard 47, then the method 100 may include, at block 170, detecting whether an internal fire 37 has occurred onboard the vehicle 10, and at block 190, detecting whether an external fire 69 has occurred outside the vehicle 10. Further, if an internal or external fire 37, 69 has been detected, then the method 100 may include, at block 210, presenting a warning 75 within the interior cabin 20 regarding the detected internal or external fire 37, 69. Additionally, if, at block 170, an internal fire 37 has been detected, then, at block 180, internal fire information 67 regarding the internal fire 37 may be received from one or more sensors 36 onboard the vehicle 10; or, if at block 190, an external fire 69 has been detected, then, at block 200, external fire information 68 regarding the external fire 69 may be received from one or more external sources 15 outside the vehicle 10. Moreover, if, at either of blocks 170 and 190, an internal or external fire 37, 69, respectively, has been detected, then, at block 280, emergency services 18 may be automatically notified of the internal or external fire 37, 69, and fire-related data 78 relating to the internal or external fire 37, 69 may be transmitted to the back office 16.

The receiving of external route information 54 (at block 470) and the presenting of the notification 57 (at block 490) may be performed if, at block 460, the air quality 50 fails to meet or exceed the air quality standard 47 for at least a predetermined or selected timespan 98. The method 100 may further include: at block 500, requesting a route selection 86 from among the one or more alternate routes 55; at block 510, receiving the route selection 86; and, at block 520, updating a navigation 85 of the vehicle 10.

According to another embodiment, a method 100 for sampling an airpath 28 within an automotive vehicle 10 includes: (i) at block 110, obtaining an air sample 34 from the airpath 28, wherein the airpath 28 flows from one or more input ports 30 to an interior cabin 20 of the vehicle 10 and then to one or more exit ports 32, wherein the one or more input ports 30 and the one or more exit ports 32 are in fluid communication with an external environment 12 outside the vehicle 10; (ii) at block 120, determining whether an air quality 50 of the air sample 34 meets or exceeds a predetermined air quality standard 47; (iii) at block 160, transmitting air quality data 52 relating to the air sample 34 to a back office 16, wherein the air quality data 52 includes one or more of a quantitative indication 59 of the air quality 50, a qualitative indication 60 of the air quality 50, and a characterization 61 of one or more components 62 found in the air sample 34; and (iv) if the air quality 50 fails to meet or exceed the air quality standard 47, then one or more of increasing a filtration level of air within the one or more input ports or within the interior cabin, increasing an odor neutralization level of air within the one or more input ports or within the interior cabin, and releasing a scent within the one or more input ports or within the interior cabin.

According to yet another embodiment, a system 42 for sampling an airpath 28 within an automotive vehicle 10 is provided. In this embodiment, the airpath 28 flows from one or more input ports 30 to an interior cabin 20 of the vehicle 10 and then to one or more exit ports 32, wherein the one or more input ports 30 and the one or more exit ports 32 are in fluid communication with an external environment 12 outside the vehicle 10. The system 42 includes: (i) a sensing module 49 configured for obtaining an air sample 34 from the airpath 28; (ii) a control module 48 in communication with the sensing module 49 and configured for determining whether an air quality 50 of the air sample 34 meets or exceeds a predetermined air quality standard 47; and (iii) a transmission module 51 in communication with the control module 48 and configured for transmitting air quality data 52 relating to the air sample 34 to a back office 16

The system may also include: (iv) a reception module 53 in communication with the control module 48 and configured for receiving external route information 54 from the back office 16 relating to one or more alternate routes 55 for the vehicle 10; and (v) a presentation module 56 in communication with the control module 48 and configured for presenting a notification 57 within the interior cabin 20 of the one or more alternate routes 55 and of a respective air quality indication 58 for each of the one or more alternate routes 55.

The control module 48 may include a processor 43 and a memory 44 in communication with the processor 43, with the memory containing an instruction set 45 operable to: cause the sensing module 49 to obtain the air sample 34 from the airpath 28; cause the processor 43 to determine whether the air quality 50 of the air sample 34 meets or exceeds the predetermined air quality standard 47; and cause the transmission module 51 to transmit air quality data 52 relating to the air sample 34 to the back office 16.

The sensing module 49 may be further configured for detecting whether an internal fire 37 has occurred onboard the vehicle 10, and the instruction set 45 may be further operable to cause the transmission module 51 to notify emergency services 18 of an internal fire 37, and cause the transmission module 51 to transmit fire-related data 78 relating to the internal fire 37 to the back office 16, if an internal fire 37 onboard the vehicle 10 has been detected.

The system 42 may further include an odor mitigation module 80 in communication with the control module 48 and configured for one or more of increasing a filtration level 81 of air within the one or more input ports 30 or within the interior cabin 20, increasing an odor neutralization level 82 of air within the one or more input ports 30 or within the interior cabin 20, and releasing a scent 41 within the one or more input ports 30 or within the interior cabin 20.

The instruction set 45 may be further operable to cause the sensing module 49 to obtain the air sample 34 when the vehicle 10 is located proximate a predetermined or indicated emission source location 17, and cause the transmission module 51 to transmit a reporting flag 99 with the air quality data 52 that relates to the air sample 34 obtained at the predetermined or indicated emission source location 17.

While various steps of the method 100 have been described as being separate blocks, and various functions of the system 42 have been described as being separate modules or elements, it may be noted that two or more steps may be combined into fewer blocks, and two or more functions may be combined into fewer modules or elements. Similarly, some steps described as a single block may be separated into two or more blocks, and some functions described as a single module or element may be separated into two or more modules or elements. Additionally, the order of the steps or blocks described herein may be rearranged in one or more different orders, and the arrangement of the functions, modules and elements may be rearranged into one or more different arrangements.

The above description is intended to be illustrative, and not restrictive. While the dimensions and types of materials described herein are intended to be illustrative, they are by no means limiting and are exemplary embodiments. In the following claims, use of the terms “first”, “second”, “top”, “bottom”, etc. are used merely as labels, and are not intended to impose numerical or positional requirements on their objects. As used herein, an element or step recited in the singular and preceded by the word “a” or “an” should be understood as not excluding plural of such elements or steps, unless such exclusion is explicitly stated. Additionally, the phrase “at least one of A and B” and the phrase “A and/or B” should each be understood to mean “only A, only B, or both A and B”. Moreover, unless explicitly stated to the contrary, embodiments “comprising” or “having” an element or a plurality of elements having a particular property may include additional such elements not having that property. And when broadly descriptive adverbs such as “substantially” and “generally” are used herein to modify an adjective, these adverbs mean “mostly”, “mainly”, “for the most part”, “to a significant extent”, “to a large degree” and/or “at least 51 to 99% out of a possible extent of 100%”, and do not necessarily mean “perfectly”, “completely”, “strictly”, “entirely” or “100%”. Additionally, the word “proximate” may be used herein to describe the location of an object or portion thereof with respect to another object or portion thereof, and/or to describe the positional relationship of two objects or their respective portions thereof with respect to each other, and may mean “near”, “adjacent”, “close to”, “close by”, “at” or the like.

This written description uses examples, including the best mode, to enable those skilled in the art to make and use devices, systems and compositions of matter, and to perform methods, according to this disclosure. It is the following claims, including equivalents, which define the scope of the present disclosure.

What is claimed is:

1. A method for sampling an airpath within an automotive vehicle, comprising:
obtaining an air sample from the airpath, wherein the airpath flows from one or more input ports to an interior cabin of the vehicle and then to one or more exit ports, wherein the one or more input ports and the one or more exit ports are in fluid communication with an external environment outside the vehicle;
determining whether an air quality of the air sample meets or exceeds a predetermined air quality standard; and
transmitting air quality data relating to the air sample to a back office.

2. The method of claim 1, further comprising:
if the air quality fails to meet or exceed the air quality standard, then:
receiving external route information from the back office relating to one or more alternate routes for the vehicle; and
presenting a notification within the interior cabin of the one or more alternate routes and of a respective air quality indication for each of the one or more alternate routes.

3. The method of claim 1, wherein the air quality data includes one or more of a quantitative indication of the air quality, a qualitative indication of the air quality, and a characterization of one or more components found in the air sample.

4. The method of claim 1, wherein the air quality data is transmitted to the back office via one or more other vehicles.

5. The method of claim 2, wherein the notification is one or both of a visual notification on a display device and an auditory notification over a speaker.

6. The method of claim 1, further comprising:
if the air quality fails to meet or exceed the air quality standard, then one or more of:
increasing a filtration level of air within the one or more input ports or within the interior cabin;
increasing an odor neutralization level of air within the one or more input ports or within the interior cabin; and
releasing a scent within the one or more input ports or within the interior cabin.

7. The method of claim 1, further comprising:
if the air quality fails to meet or exceed the air quality standard, then automatically closing any open windows of the vehicle.

8. The method of claim 1, further comprising:
if the air quality fails to meet or exceed the air quality standard, then detecting whether an internal fire has occurred onboard the vehicle or an external fire has occurred outside the vehicle; and
if an internal or external fire has been detected, then presenting a warning within the interior cabin regarding the detected internal or external fire.

9. The method of claim 8, further comprising:
if an internal fire has been detected, then receiving internal fire information regarding the internal fire from one or more sensors onboard the vehicle, or if an external fire has been detected, then receiving external fire information regarding the external fire from one or more external sources outside the vehicle.

10. The method of claim 8, further comprising:
if an internal or external fire has been detected, then automatically notifying emergency services of the internal or external fire; and
transmitting fire-related data relating to the internal or external fire to the back office.

11. The method of claim 2, wherein the receiving and presenting are performed if the air quality fails to meet or exceed the air quality standard for at least a predetermined or selected timespan.

12. The method of claim 11, further comprising:
requesting a route selection from among the one or more alternate routes;
receiving the route selection; and
updating a navigation of the vehicle.

13. The method of claim 1, wherein the obtaining of the air sample is performed when the vehicle is located proximate a predetermined or indicated emission source location, and further comprising:
transmitting a reporting flag with the air quality data that relates to the air sample obtained at the predetermined or indicated emission source location.

14. A method for sampling an airpath within an automotive vehicle, comprising:
obtaining an air sample from the airpath, wherein the airpath flows from one or more input ports to an interior cabin of the vehicle and then to one or more exit ports, wherein the one or more input ports and the one or more exit ports are in fluid communication with an external environment outside the vehicle;
determining whether an air quality of the air sample meets or exceeds a predetermined air quality standard;
transmitting air quality data relating to the air sample to a back office, wherein the air quality data includes one or more of a quantitative indication of the air quality, a qualitative indication of the air quality, and a characterization of one or more components found in the air sample; and
if the air quality fails to meet or exceed the air quality standard, then one or more of:
increasing a filtration level of air within the one or more input ports or within the interior cabin;
increasing an odor neutralization level of air within the one or more input ports or within the interior cabin; and
releasing a scent within the one or more input ports or within the interior cabin.

15. A system for sampling an airpath within an automotive vehicle, wherein the airpath flows from one or more input ports to an interior cabin of the vehicle and then to one or more exit ports, wherein the one or more input ports and the one or more exit ports are in fluid communication with an external environment outside the vehicle, the system comprising:
a sensing module configured for obtaining an air sample from the airpath;
a control module in communication with the sensing module and configured for determining whether an air quality of the air sample meets or exceeds a predetermined air quality standard; and
a transmission module in communication with the control module and configured for transmitting air quality data relating to the air sample to a back office.

16. The system of claim 15, further comprising:
a reception module in communication with the control module and configured for receiving external route information from the back office relating to one or more alternate routes for the vehicle; and
a presentation module in communication with the control module and configured for presenting a notification within the interior cabin of the one or more alternate routes and of a respective air quality indication for each of the one or more alternate routes.

17. The system of claim 15, wherein the control module includes a processor and a memory in communication with the processor, the memory containing an instruction set operable to:
cause the sensing module to obtain the air sample from the airpath;
cause the processor to determine whether the air quality of the air sample meets or exceeds the predetermined air quality standard; and
cause the transmission module to transmit air quality data relating to the air sample to the back office;

18. The system of claim 17, wherein the sensing module is further configured for detecting whether an internal fire has occurred onboard the vehicle and the instruction set is further operable to:
if an internal fire onboard the vehicle has been detected, then:
cause the transmission module to notify emergency services of the internal fire; and
cause the transmission module to transmit fire-related data relating to the internal fire to the back office.

19. The system of claim 15, further comprising:
an odor mitigation module in communication with the control module and configured for one or more of:
increasing a filtration level of air within the one or more input ports or within the interior cabin;
increasing an odor neutralization level of air within the one or more input ports or within the interior cabin; and
releasing a scent within the one or more input ports or within the interior cabin.

20. The system of claim 17, wherein the instruction set is further operable to:
cause the sensing module to obtain the air sample when the vehicle is located proximate a predetermined or indicated emission source location; and
cause the transmission module to transmit a reporting flag with the air quality data that relates to the air sample obtained at the predetermined or indicated emission source location.
